(19) United States
(12) Patent Application Publication
(10) Pub. No.: US 2015/0056140 A1
(43) Pub. Date: Feb. 26, 2015

(54) ELECTROLYTE PURGATIVES

(71) Applicants: Thomas Julius Borody, Five Dock, NSW (AU); Sanjay Ramrakha, Five Dock, NSW (AU); John Saxon, Five Dock, NSW (AU); Antony Wettstein, Five Dock, NSW (AU)

(72) Inventors: Thomas Julius Borody, Five Dock (AU); Sanjay Ramrakha, Five Dock (AU); John Saxon, Five Dock (AU); Antony Wettstein, Five Dock (AU)

(21) Appl. No.: 14/353,744

(22) PCT Filed: Oct. 27, 2012
(86) PCT No.: PCT/AU2012/001315

§ 371 Date: Apr. 23, 2014

Related U.S. Application Data
(60) Provisional application No. 61/552,431, filed on Oct. 27, 2011.
(60) Provisional application No. 61/717,599, filed on Oct. 23, 2012.

(51) Int. Cl. A61K 31/538; A61K 47/02; A61K 9/48; A61K 47/10

(52) U.S. Cl. CPC A61K 31/538; A61K 47/10; A61K 47/02; A61K 9/48
USPC 424/9.1; 514/230.5

(57) ABSTRACT

The invention provides compositions for use in purgatives, for use as purgatives, and to methods for inducing purgation of the colon. In alternative embodiments, the invention provides compositions, e.g., a purgative, comprising: a water-soluble sodium salt (especially sodium sulphate), a water-soluble potassium salt (especially potassium sulphate), and a water-soluble magnesium salt (especially magnesium sulphate); and, compositions further comprising: a bisoxatin, or a detergent stool softening agent (such as sodium picosulphate) and/or a water-soluble sugar (such as xylose or equivalent); or, compositions having these ingredients at different amounts, but at equivalent proportions. In alternative embodiments, the invention provides purgative compositions comprising electrolytes, salts, sugars, bisoxatin, dyes and biofilm disruptors.

### FIELD OF THE INVENTION

The invention provides compositions for use in purgatives, for use as purgatives, and to methods for inducing purgation of the colon. In alternative embodiments, the invention provides compositions, e.g., a purgative, comprising: a sodium sulphate, a potassium sulphate, and a magnesium sulphate; and, compositions further comprising: a bisoxatin, or a sodium picosulphate and/or a xylose or equivalent; or, compositions having these ingredients at different amounts, but at equivalent proportions. In alternative embodiments, the invention provides purgative compositions comprising electrolytes, salts, sugars, bisoxatin, dyes and biofilm disruptors.

### BACKGROUND ART

Colonic orthostatic lavage is an iatrogenic phenomenon related to the administration of a purgative and therefore is predictable in its action and side effects. It is important to make the distinction between the use of iatrogenic purgation solutions and fluid/electrolyte replacement solutions used for treatment of vomiting and diarrhea associated with gastroenteritis. The use of mainly hypotonic or isotonic solutions such as glucose-based ‘Bangladesh’ solution and rice-based solutions has been successful in patients with gastroenteritis and dehydration, a highly unpredictable disease. The physiological principle of coupled sodium and glucose transport in a 1:1 molar ratio in the intestine has been shown to be safe and effective.

Purgatives developed to date for orthostatic lavage to clean the bowel of faecal matter prior to colonoscopy have taken the form of either an isotonic, large volume lavage (e.g. Braintree's Golytely) or more hypertonic lavage products such as Fleet's sodium phosphate or sodium picosulfate (Picolax) products. The former generally cause little homeostatic disturbance of intra-vascular sodium and other electrolytes or fluid shifts because of their isotonic nature, which minimizes electrolyte absorption/secretion by the presence of high molecular weight polyethylene glycol (PEG mw 3350). However, these preparations have recently been reported to be associated with hyponatremia (Cohen D. C. et al., Lancet 357(9252): 282-283 (2001)). Products with sodium phosphate and sodium picosulfate are felt to be better tolerated (Fincher R. K., et al., Am. J. Gastroenterol. 94(8): 2122-7 (1999)). However, these products have also been associated with a significant hypo-osmolar state and electrolyte imbalance, particularly hyponatremia. This, to a large extent, is contributed to by a loss of electrolytes through the resultant diarrhea caused by the lavage with concomitant replacement of this loss by water (without electrolytes) leading to hyponatremia and water intoxication associated with a hypo-osmolar state.

The symptoms of headache, lethargy and nausea reported by patients undergoing orthostatic lavage are felt to be due to an osmotic shift with resultant dilutional hyponatremia that is induced by the various bowel preparation products such as “Fleet”, Picolax etc. This effect appears to be more pronounced in adult females, perhaps as a result of relatively less total body water when compared to adult males and children (Fraser et al., Am. J. Physiol. 256: R880-5 (1989)).

The clinical features of hyponatremia (hypoosmolality) are highly variable and their severity correlates poorly with the level of serum sodium. Classically, the clinical features of severe hyponatremia are confusion, seizures and obtundation.

A decrease in plasma osmolality causes brain swelling (cerebral edema) as water moves along osmotic gradients. In response, the brain loses solute from the intra- and extra-cellular fluid spaces, which returns brain water content back towards normal. Once the brain has equilibrated (i.e. volume-adapted) through solute losses, neurological features will be less prominent or resolve.

The rate of fall of serum osmolality is generally better correlated with morbidity and mortality than the actual magnitude of the decrease (Arieff, A. I. et al., Medicine (Baltimore) 55: 121-9 (1976)), and is somewhat arbitrarily defined as hypoosmolality developing over 24 to 48 hours. Mortality up to 50% has been reported in patients with acute hyponatremia (Arieff, A. I. et al., loc.cit.). Cerebral edema develops when hypoosmolality exceeds the ability of the brain to regulate its volume by solute losses. In experimental models, acute hyponatremia results in the loss of sodium and chloride from the brain within 30 minutes, whilst potassium loss is more delayed. All electrolyte losses are maximal by 3 hours after initiation of hyponatremia (Melton, J. E. et al., Am. J. Physiol. 252: F661-9 (1987)).

Hence in some situations the effects of the various bowel purgative formulations currently available can lead to the unpleasant side effects of headache, malaise and dizziness and hypotension. Additionally, life threatening presentations of hypo-osmolar grand mal epileptic seizures, asphyxia and death have been reported.

Due to the accepted benefits of screening colonoscopic surveillance programs for the detection of colonic polyps and bowel cancer, the utilization of colonic lavage is increasing rapidly. Indeed it is feasible that a large number of the population over the age of 50 years is likely to undergo colonoscopic examination. As a result, a considerable number of patients could potentially develop lavage-related hyponatremia and hypo-osmolar water intoxication with subsequent ‘dilution’ of other electrolytes leading to significant morbidity and potentially mortality.

Poor palatability leading to reduced patient compliance has been an important issue in the failure of some of the currently available products; either the volume is too large or the taste too objectionable for certain patients to comply with taking the prescribed bowel preparation. This leads to inadequate orthostatic lavage causing poor visibility at colonoscopy.

There is therefore a need for a purgative composition that reduces mortality and/or patient morbidity and/or which makes the procedure of purgation of the colon much more pleasant for the patient so as to facilitate patient compliance.

### SUMMARY OF THE INVENTION

In alternative embodiments, the invention provides compositions, pharmaceutical compositions or formulations (e.g., as a purgative), comprising:

at least one water-soluble sodium salt,

at least one water-soluble potassium salt;

at least one water-soluble sugar, or a water-soluble degradable sugar, or alternatively, a minimally degradable sugar;

a detergent stool softening agent;

and a bisoxatin (or 2,2-bis(4-hydroxyphenyl)-2H-benzo[b][1,4]oxazin-3(4H)-one), or bisoxatin acetate, or equivalent, including e.g., a LAXONALIN™, a MARATAN™, a TALSIS™, or a TASIS™, or an equivalent. In alternative embodiments, a formulation or composition of the invention comprises between about 10 mg to about 0.5, 1, 2, 2.5, 3, 3.5, 4, 4.5 or 5 or more grams of bisoxatin, or between about 0.5 and 5 grams (g) of bisoxatin, or between about 75, 80, 85, 90 or 100 mg to about 150 to 200 mg (e.g., for a normal patient) bisoxatin, or between about 100, 110, 120, 130, 140 or 150 mg to about 1, 2, 3, 4, 4.5 or 5 grams (g) or more bisoxatin (e.g., for a constipated patient).

In alternative embodiments, the invention provides compositions, pharmaceutical compositions or formulations (e.g., as a purgative), comprising
(a) (i) 1 to about 10 gram per unit dose, or between about 1 to 10 gram per unit dose, or about 0.5, 1, 2, 3, 4, 5, 6, 7, 8, 9, 10, 11, 12, 13, 14, 15, 16, 17, 17.5, 18, 19 or 20 or more gram per unit dose, of at least one water-soluble sodium salt;
(ii) 1 or 2 to about 20 gram per unit dose, or between about 1 to 20 gram per unit dose, or about 0.5, 1, 2, 3, 4, 5, 6, 7, 8, 9, 10, 11, 12, 13, 14, 15, 16, 17, 18, 19 or 20 or more gram per unit dose, of at least one water-soluble sugar, or a water-soluble degradable sugar, or alternatively, a minimally degradable sugar;
(iii) 0.5 to about 5 gram per unit dose, or between about 0.5 to 10 gram per unit dose, or about 0.1, 0.2, 0.3, 0.4, 0.5, 1.0, 2, 3, 3.1, 3.2, 3.3, 3.4, 3.5, 4, 5, 6, 7, 8, 9, or 10 or more gram per unit dose, of at least one water-soluble potassium salt;
(iv) 1 to about 10 gram per unit dose, or between about 1 to 10 gram per unit dose, or about 0.1, 0.2, 0.3, 0.4, 0.5, 0.6, 0.7, 0.8, 0.9, 1.0, 2, 3, 4, 5, 6, 7, 8, 9 or 10 or more gram per unit dose, of at least one water-soluble magnesium salt; and
(v) a detergent stool softening agent;

wherein the composition is a hypertonic composition, optionally in the form of a unit dose having a volume of from about 0.2 to about 0.5 liter (L), or dose having a volume of about 0.1, 0.2, 0.3, 0.4, 0.5, 0.6 or more L,

and wherein optionally the sugar, or the degradable sugar, or the minimally degradable sugar, comprises a xylose, a xylotriose, a mannitol, a xylooligosaccharide, a fructooligosaccharide, a fructosan, a galactooligosaccharide, an equivalent degradable sugar thereof or a mixture thereof;
(b) the composition, pharmaceutical composition, or formulation, of (a), wherein the composition is a purgative or a purgative composition;
(c) the composition of (a) or (b), wherein the composition comprises:

a sodium sulphate at a per unit dose of about 17.5 gram (g), or between about 2 to about 37 gram,

a potassium sulphate at a per unit dose of about 3.13 g, or between about 0.1 to about 4.8 g, and

a magnesium sulphate at a per unit dose of about 1.6 g, or between about 0.1 to about 7 g;
(d) the composition of any of (a) to (c), wherein the composition further comprises:
   a sodium picosulphate at a per unit dose of about 30 mg, or between about 0.01 to about 100 mg, and/or
   a xylose at a per unit dose of about 7.5 g, or between about 3 to about 15 g;
(e) the ingredients of any of (a) to (d) at equivalent proportions; or
(f) the composition of any of (a) to (e), further comprising a bisoxatin (or 2,2-bis(4-hydroxyphenyl)-2H-benzo[b][1,4]oxazin-3(4H)-one), or bisoxatin acetate, or equivalent,

wherein optionally the bisoxatin is a LAXONALIN™, a MARATANT™, a TALSIS™, or a TASIS™, or an equivalent,

and optionally the composition, pharmaceutical composition, formulation comprises between about 10 mg to about 0.5, 1, 2, 2.5, 3, 3.5, 4, 4.5 or 5 or more grams of bisoxatin, or between about 0.5 and 5 grams (g) of bisoxatin, or between about 75, 80, 85, 90 or 100 mg to about 150 to 200 mg bisoxatin, or between about 100, 110, 120, 130, 140 or 150 mg to about 1, 2, 3, 4, 4.5 or 5 grams (g) or more bisoxatin.

In alternative embodiments, the invention provides compositions, pharmaceutical compositions or formulations, comprising:
(a) (i) at least one water-soluble sodium salt;
   (ii) at least one water-soluble minimally degradable sugar or oligosaccharide in an amount, wherein the total weight of water-soluble minimally degradable sugar or oligosaccharide in the composition is from about 1 to about 3 times the weight of the sodium salt in the composition;
   (iii) at least one water-soluble potassium salt, wherein the weight of the water-soluble potassium salt in the composition is from about 0.05 to about 1 times the weight of the sodium salt in the composition; and
   (iv) at least one water-soluble magnesium salt, wherein the weight of magnesium salt in the composition is from about 0.1 to about 10 times the weight of the sodium salt in the composition; and
   (v) a detergent stool softening agent,

wherein optionally the minimally degradable sugar or oligosaccharide comprises a mannitol, a xylose, a xylotriose, a xylooligosaccharide, a fructooligosaccharide, a fructosan, a galactooligosaccharide, an equivalent minimally degradable sugar or oligosaccharide or a mixture thereof,

and wherein the purgative composition is formulated as a hypertonic composition in the form of a unit dose;
(b) the composition of (a), wherein the composition is a purgative or a purgative composition;
(c) the composition of (a) or (b), wherein the composition comprises:
   a sodium sulphate at a per unit dose of about 17.5 gram (g), or between about 2 to about 37 gram,
   a potassium sulphate at a per unit dose of about 3.13 g, or between about 0.1 to about 4.8 g, and
   a magnesium sulphate at a per unit dose of about 1.6 g, or between about 0.1 to about 7 g;
(d) the composition of any of (a) to (c), wherein the composition further comprises:
   a sodium picosulphate at a per unit dose of about 30 mg, or between about 0.01 to about 100 mg, and/or
   a xylose at a per unit dose of about 7.5 g, or between about 3 to about 15 g;
(e) the ingredients of any of (a) to (d) at equivalent proportions; or
(f) the composition of any of (a) to (e), further comprising a bisoxatin (or 2,2-bis(4-hydroxyphenyl)-2H-benzo[b][1,4]oxazin-3(4H)-one), or bisoxatin acetate, or equivalent,

wherein optionally the bisoxatin is a LAXONALIN™, a MARATAN™, a TALSIS™, or a TASIS™, or an equivalent,

and optionally the composition, pharmaceutical composition, formulation comprises between about 10 mg to about 0.5, 1, 2, 2.5, 3, 3.5, 4, 4.5 or 5 or more grams of bisoxatin, or between about 0.5 and 5 grams (g) of bisoxatin, or between about 75, 80, 85, 90 or 100 mg to about 150 to 200 mg bisoxatin, or between about 100, 110, 120, 130, 140 or 150 mg to about 1, 2, 3, 4, 4.5 or 5 grams (g) or more bisoxatin.

In alternative embodiments, the water-soluble sodium salt is selected from the group consisting of sodium sulphate, a sodium chloride, a sodium gluconate, a sodium citrate, a sodium aspartate and mixtures thereof; or, wherein the water-soluble potassium salt is selected from the group consisting of a potassium sulfate, a potassium chloride and a potassium tartrate; or wherein the water-soluble magnesium salt is selected from the group consisting of a magnesium sulfate, a magnesium citrate and a magnesium phosphate and mixtures thereof.

In alternative embodiments, the detergent stool softening agent is a sodium picosulfate, a sodium sulphate, a bisacodyl or a combination thereof.

In alternative embodiments, the compositions pharmaceutical compositions or formulations further comprise at least one composition or additive selected from the group consisting of a flavoring ingredient, citrate, lactate, acetate, a trace element and a nutritional element.

In alternative embodiments, compositions pharmaceutical compositions or formulations of the invention are in the form of, or formulated as a liquid, a fluid, a soup or soup-like composition, tablet, gel cap, capsule or sachet.

In alternative embodiments, compositions pharmaceutical compositions or formulations of the invention are in the form of a unit dose having a volume of from about 0.1 to 1.0 L and wherein:

the sodium salt or salts are present in an amount from about 1 to about 20 g per unit dose, or at about 0.5, 1, 2, 3, 4, 5, 6, 7, 8, 9, 10, 11, 12, 13, 14, 15, 16, 17, 17.5, 18, 19 or 20 or more per unit dose;

the minimally degradable sugar or sugars in an amount of from about 1 or 2 to about 20 or more g, or at about 0.5, 1, 2, 3, 4, 5, 6, 7, 8, 9, 10, 11, 12, 13, 14, 15, 16, 17, 17.5, 18, 19 or 20 or more g per unit dose;

the potassium salt or salts in an amount of from about 0.5 to about 5 g, or at about 0.5, 1, 2, 3, 4, 5, 6, 7, 8, 9, 10, 11, 12, 13, 14, 15, 16, 17, 17.5, 18, 19 or 20 or more or more g per unit dose;

the magnesium salt or salts in an amount of from about 1 to about 20 g per unit dose of the purgative composition, or at about 0.5, 1, 2, 3, 4, 5, 6, 7, 8, 9, 10, 11, 12, 13, 14, 15, 16, 17, 17.5, 18, 19 or 20 or more or more g per unit dose.

In alternative embodiments:
(i) the at least one water-soluble sodium salt comprises a sodium sulfate or a sodium chloride;
(ii) the at least one water-soluble minimally degradable sugar comprises a xylose;
(iii) the at least one water-soluble potassium salt comprises a potassium sulfate or a potassium chloride; or
(iv) the at least one water-soluble magnesium salt comprises magnesium sulfate.

In alternative embodiments, compositions of the invention further comprise one or more compositions or additives selected from the group consisting of a citrate, a lactate, an acetate, a calcium, a zinc, a Vitamin B complex, a thiamine, a Vitamin A, a Vitamin C, a Vitamin E, a folic acid and a biotin.

In alternative embodiments, the invention provides compositions pharmaceutical compositions or formulations in the form of:
(a) a tablet or capsule, or
(b) a tablet or capsule comprising:
   a core comprising the sodium, potassium and magnesium salts; and
   a coating comprising the minimally degradable sugar(s);
wherein the coating surrounds the core or capsule content.

In alternative embodiments, the invention provides compositions pharmaceutical compositions or formulations wherein:

the at least one water-soluble sodium salt comprises a sodium sulfate, a sodium chloride, a sodium gluconate, a sodium citrate or a sodium aspartate;

the at least one water-soluble potassium salt comprises a potassium sulfate, or a potassium chloride; or

the at least one water-soluble magnesium salt comprises a magnesium sulfate, a magnesium citrate or a magnesium phosphate.

In alternative embodiments, the invention provides methods of inducing a pre-surgical lavage of the colon of a patient in need thereof, comprising administering to the patient a purgative composition of the invention, in an amount effective for pre-surgical lavage of the patient's colon.

In alternative embodiments, the invention provides methods of inducing purgation of the colon of a patient in need thereof, comprising administering to the patient a purgative composition of the invention, in an amount effective to induce purgation of the patient's colon.

In alternative embodiments, the invention provides pharmaceutical compositions or formulations or purgative compositions comprising:
(a) a sodium sulphate at a per unit dose of about 17.5 gram (g), or between about 2 to about 37 gram,
   a potassium sulphate at a per unit dose of about 3.13 g, or between about 0.1 to about 4.8 g, and
   a magnesium sulphate at a per unit dose of about 1.6 g, or between about 0.1 to about 7 g;
(b) the composition of (a), wherein the composition further comprises:
   a sodium picosulphate at a per unit dose of about 30 mg, or between about 0.01 to about 100 mg, and/or
   a xylose at a per unit dose of about 7.5 g, or between about 3 to about 15 g; or
(c) the ingredients of (a) or (b) at equivalent proportions.

In alternative embodiments, the invention provides pharmaceutical compositions or formulations or purgative compositions comprising:
(a) a sodium sulphate at a per unit dose of about 17.5 gram (g),
   a potassium sulphate at a per unit dose of about 3.13 g, and
   a magnesium sulphate at a per unit dose of about 1.6 g;
(b) the composition of (a), wherein the composition further comprises:
   a sodium picosulphate at a per unit dose of about 30 mg, and/or
   a xylose at a per unit dose of about 7.5 g, or between about 3 to about 15 g; or
(c) the ingredients of (a) or (b) at equivalent proportions.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DETAILED DESCRIPTION OF THE INVENTION

In alternative embodiments, the invention provides purgative compositions comprising electrolytes, salts, sugars, bisoxatin, dyes and biofilm disruptors. In alternative embodiments, the invention provides purgative compositions comprising electrolytes, salts, sugars, s bisoxatin, dyes, lubricants and biofilm disruptors. In alternative embodiments, the invention provides purgative compositions comprising electrolytes, salts, sugars, and dyes and optionally biofilm disruptors, bisoxatin and/or lubricants.

In alternative embodiments, the invention provides compositions that can be used as purgatives, e.g., compositions comprising: a sodium sulphate at a per unit dose of about 17.5 gram (g), or between about 2 to about 37 gram, a potassium sulphate at a per unit dose of about 3.13 g, or between about 0.1 to about 4.8 g, and a magnesium sulphate at a per unit dose of about 1.6 g, or between about 0.1 to about 7 g; or, compositions further comprising:

a sodium picosulphate at a per unit dose of about 30 mg, or between about 0.01 to about 100 mg, and/or a xylose at a per unit dose of about 7.5 g, or between about 3 to about 15 g; or compositions having these ingredients at different amounts, but at equivalent proportions.

In alternative embodiments, the combined effects of the water-soluble sodium, potassium and magnesium salts and the minimally degradable sugar(s) in the compositions and purgatives of the invention cause a purgative effect which is surprisingly greater than the effect that would have been expected from the known effects of the same amounts of the individual components of the compositions. That is, the amounts of the salts required for simply performing their known purgative function would be significantly greater if they were used singly.

In alternative embodiments, other benefits of the compositions and purgatives of the present invention are not provided by compositions of only a single component. In alternative embodiments the increased tonicity of compositions of the invention compared to existing products enables a reduction in the amount of each constituent while maintaining the desired purgative effect. In alternative embodiments, the components of the purgatives of the invention cooperate to provide a purgative which is palatable and which causes purgation without the side effects seen with prior art compositions, in a way that could not have been predicted prior to the present invention.

In alternative embodiments, the invention provides formulations, which safely achieve orthostatic bowel lavage without associated hypo-osmolar hyponatremia. In alternative embodiments, the formulations of the invention can achieve rapid resolution and symptom reversal together with electrolyte replacement in certain infective conditions of the gastrointestinal tract. In alternative embodiments, the compositions of the invention may also be used for patients with either acute or chronic constipation, since their purgative effect, secondary to combined hypertonic effect, is not associated with melanosis seen particularly in patients taking senna-containing faecal softening agents.

In alternative embodiments, the additional function of the compositions is to combine sugar and sodium in amounts that assist in transluminal absorption of sodium and water. Individually, oral rehydration solutions (compositions) utilize this principle. In alternative embodiments, the compositions of the present invention have the unique and surprising feature of causing a purgative effect while performing the function of assisting in transluminal absorption of sodium and water.

While the invention is not limited by any particular mechanism of action, the administration of a hyperosmolar sodium load together with other electrolytes and sugar(s) and optionally trace elements at a time when the maximum effect of the iatrogenic purgative occurs reduces the gradient of change in serum osmolarity. In alternative embodiments the compositions of the invention prevent or mitigate osmolar and sodium shifts and cause a reduction in the undesirable side effects, e.g., as those seen with administration of prior art purgatives, as noted above.

In alternative embodiments, the expression “minimally degradable sugar” is to be understood to mean a carbohydrate moiety that is substantially resistant to endogenous digestion in the gastrointestinal tract.

In alternative embodiments of compositions of the invention, the minimally degradable sugar is a xylose or a xylotriose or equivalent. In alternative embodiments, other sugars including oligosaccharides such as other xylooligosaccharides, fructooligosaccharides, fructosans, galactooligosaccharides and the like are be used.

Glucose and other complex sugars used in standard oral rehydration therapy lead to intestinal decomposition with the formation of gases such as methane and hydrogen which have been associated with explosion caused by diathermy (Altomare D. F. et al., Dis Colon Rectum 36: 291-2 (1993)). In alternative embodiments, the use of minimally degradable sugars in the compositions of the present invention prevents this from occurring and reduces the incidence of abdominal cramps. In situations however where diathermy is not to be used, the minimally degradable sugar can be replaced in the compositions of the invention with a degradable sugar such as glucose, L-glucose, sucrose, fructose, galactose or lactose.

In alternative embodiments, the use of xylose (or other minimally degradable sugars) allows for transport of sodium into the alimentary cellular structure. In alternative embodiments, the combination of xylose and sodium salts thus allows for replacement of electrolytes from the induced faecorrhoea, in particular sodium, potassium and chloride, and reduces the dilutional hyponatremia associated with other products such as Picoprep, Fleet and recently reported with polyethylene glycol.

In alternative embodiments, the water-soluble sodium salt is selected from the group consisting of sodium chloride, sodium gluconate, sodium citrate and sodium aspartate.

In alternative embodiments, they include at least one sodium salt other than sodium chloride, more preferably sodium gluconate, sodium citrate or sodium aspartate, which reduce the salty taste.

In alternative embodiments, the water-soluble potassium salt is selected from the group consisting of potassium chloride and potassium tartrate. In alternative embodiments, the ratio of potassium salt(s) to sodium salt(s) in the compositions of the invention is from about 1:1 to about 1:8, more usually from about 1:1.5 to about 1:6, still more usually from about 1:2 to about 1:5, even more usually about 1:3, on a weight basis.

In alternative embodiments, the water-soluble magnesium salt is selected from the group consisting of magnesium sulfate, magnesium citrate and magnesium phosphate. Usually, the ratio of the weight of magnesium ions to the weight of sodium ions in the compositions of the invention is from about 1:5 to about 5:1, more usually from about 1:3 to about 3:1, still more usually from about 1:2 to about 2:1, even more usually about 1:1.

In alternative embodiments, the sodium salt or salts is/are typically present in an amount ranging from about 1-10 g, more typically about 5 g per unit dose of the purgative, which will usually be a volume of from about 0.2 to 0.5 L.

In alternative embodiments, the compositions of the invention comprise sodium chloride, potassium chloride, magnesium sulfate, and xylose or other minimally degradable sugars.

In alternative embodiments, compositions of the invention may be used for colonoscopic lavage, as a simple purgative or in electrolyte replacement therapy. The composition may be used with one or more known purgatives and in that case will complement the purgative effect of the other purgative(s) and thus reduce the amount required of these purgative agents. For example a composition of the present invention may be administered with a half dose of Fleet, or a reduced number of Picoprep capsules.

In alternative embodiments, the composition further comprise one or more further additives selected from citrate, lactate, acetate, trace elements such as calcium and zinc, nutritional elements such as Vitamin B complex, thiamine, Vitamin A, Vitamin C, Vitamin E, folic acid, and biotin. These additives may be included in the compositions of the invention in amounts which are based on the patient's daily dietary requirements.

In alternative embodiments, the ratio of minimally degradable sugar(s) to sodium ions in the compositions and purgatives of the invention is from about 3:1 to 1:1 on a weight basis, and will more typically be about 2:1 to 1.4:1. The minimally degradable sugar or sugars is/are typically present in an amount ranging from about 2 to 20 g, more typically about 10 g per unit dose.

In alternative embodiments, the potassium salt or salts is/are typically present in an amount ranging from about 0.5 to 5 g per unit dose, more typically about 1 to 5 g per unit dose, still more typically about 1.5 to 3 g per unit dose.

In alternative embodiments, the magnesium salt or salts is/are typically present in an amount ranging from about 1 to about 10 g per unit dose, more typically about 3 to 5 g per unit dose.

In alternative embodiments, the sodium is present at a concentration of from about 200-700 milliosmole (mosm). More typically, the purgative includes sodium at about three times the isotonic concentration (that is, about 270 mosm).

In the methods of the third embodiment, the composition of the invention is typically administered in an amount sufficient to provide to the patient the following quantities of the components:
(i) sodium in an amount of from about 0.01 to about 1.5 g per kg body weight, more usually about 0.05 to about 1 g per kg, still more usually about 0.08 g per kg, in which case the administered dose of sodium will approximate 5 g for an individual weighing 60-70 kg;
(ii) the minimally degradable sugar or sugars in an amount of from about 0.02 to about 3 g per kg of body weight, more usually from about 0.1 to about 0.2 g per kg, still more usually about 0.15 g per kg in which case the administered dose of minimally degradable sugar will approximate 10 g for an individual weighing 60-70 kg;
(iii) potassium in an amount of from about 0.005 to about 0.1 g per kg body weight, more usually from about 0.01 to about 0.05 g per kg, still more usually about 0.03 g per kg in which case the administered dose approximates 2 g for an individual weighing 60-70 kg;
(iv) magnesium in an amount of from about 0.01 to about 1.5 g per kg body weight, more usually about 0.05 to about 1 g per kg, still more usually about 0.08 g per kg in which case the administered dose approximates 5 g for an individual weighing 60-70 kg.

In alternative embodiments, following the oral ingestion of the purgative of the invention, cool water in a volume greater than three times the volume of the purgative hypertonic solution is ingested.

In alternative embodiments, the compositions of the invention further comprise a detergent stool-softening agent such as sodium picosulfate. In alternative embodiments, this is in an amount of from about 5 to about 25 mg; or from about 10-15 mg, per unit dose of the composition.

The purgative of the second embodiment may suitably be prepared by dissolving a required amount of a composition of the first embodiment in a suitable quantity (typically from about 200 mL to 500 mL) of cold, warm or hot water.

In other forms the composition of the invention may be compressed into tablets, gel caps or capsules. In this form it is useful for pre-colonoscopic orthostatic lavage of the bowel, as preparation for barium enema, in CT “virtual colonoscopy” and for other radiological applications. It is also useful in pre-surgical lavage e.g. for removal of the bowel for cancer, diverticulitis etc. When formulated as tablets, the tablets may suitably comprise a core of the sodium, potassium and magnesium salts, surrounded by a coating of the minimally degradable sugar(s).

The composition or purgative of the invention may further comprise at least one flavoring ingredient, such as chicken, beef, vegetarian, Thai, seafood, spice or curry. Suitably, the purgative of the second embodiment is formulated as a soup or soup-like composition.

The psychological advantage of an easily tolerated fluid with versatility of flavors is that it may be substituted for a meal for patients who are on a restricted low residue clear fluids regime. In alternative embodiments, the invention uses various flavors such as chicken, beef, vegetable, kosher, gluten free, Thai, Japanese (teriyaki), Indian (curry) etc in a soup mix which includes a composition of the first embodiment and which allows for individual preference.

In alternative embodiments, if the purgative of the invention is administered as a clear soup, the purgative is made up using hot water rather than cool fluids. Improved tolerance and compliance is thereby achieved, in part by reducing the volume of the preparation to 350 ml and in part by providing a hypertonic “tasty” meal, as opposed to 3 liters of an unpalatable isotonic solution such as polyethylene glycol.

In alternative embodiments, the purgative of the invention is an electrolyte replacement product, which may accompany and augment the action of other purgative agents such as products containing sodium picosulfate and sodium phosphate (e.g. Fleet and Picolax/Picoprep). In alternative embodiments, the purgative of the invention, when administered in an effective amount to a patient, contributes to lavage but leads to fewer complications such as hyponatremia, and hypoosmolar dilutional state, and to fewer symptoms such as dizziness, nausea, headache and hypotension, than known purgative agents.

Although the ratio of individual salts in the compositions of the invention may vary within the ranges stated above, it is the combination of these salts added to a defined volume of water which forms a hypertonic salt solution. The tonicity of fluid is the key to the electrolyte replacement and purgative effect of the purgatives of the invention.

In alternative embodiments, part of the preparation involves an intact thirst mechanism which is provided by the hypertonic load, patients for whom administration of compositions of the invention is to be used with caution include the very young, the infirmed and demented, those unable to self-administer water or other fluids, and those patients in which a large sodium load is undesirable (that is, patients with LVEF <25%), renal failure patients, those with advanced cardiac or renal disease and those with pituitary adenoma/hypofunction.

In alternative embodiments, the compositions comprise an electrolyte replacement lavage solution, which can have several roles. In alternative embodiments, it can be administered with hyper-osmolar solutions such as products containing sodium picosulfate and sodium phosphate (e.g. Fleet and Picolax/Picoprep). It can also be used as an electrolyte replacement lavage solution for acute gastrointestinal infections including salmonella, Shigella, campylobacter or viral gastroenteritis. This is applicable in particular to viral gastritis or bacterial gastroenteritis so as to give patient's a clearance of contents of the flora as well as replace electrolytes that are being lost during the gastroenteritis. It can also provide symptomatic improvement in those patients suffering from acute or chronic constipation and related symptoms and for those with constipation predominant irritable bowel syndrome (IBS). In addition, the product can be used alone as an effective orthostatic lavage for the following applications: prior to colonoscopy, CT scanning “virtual colonoscopy”, barium enema examination, or intestinal surgery. This is due to the product allowing simultaneous lavage of the bowel and replacement of essential electrolytes with fewer complications such as hyponatremia, hypo-osmolar dilutional state, and fewer symptoms such as dizziness, nausea and headache.

In alternative embodiments, the effective hypertonicity of the purgatives of the invention will cause purgation when administered to a patient undergoing a procedure for which purgation is required. These patients adhere to bowel preparation protocols which commonly instruct a low residue diet and clear fluids for 1 to 2 days prior to the procedure for which they are being prepared. In administering the purgatives of this invention a smaller volume (approximately 200-500 ml) of hyperosmolar electrolyte enhanced fluid is required as opposed to larger volumes (3-4 liters) of isotonic balanced salt solution (OLYCOPREP™). The patients continue to consume clear fluids to maintain hydration. This is more palatable and acceptable to the patient. The volume of the purgatives of the present invention is much less (typically about one tenth) of the volume of solutions of prior art purgatives which are administered to a patient. Other fluid taken is part of a normal diet, and hence is better tolerated and more palatable, with better patient compliance.

In alternative embodiments, the compositions and purgatives of the invention are particularly useful for constipation and bloating, and as soup-like preparations the purgatives of the invention are acceptable to patients as a daily food product. As a flavored medication they have particular use as simultaneous orthostatic lavage and electrolyte replacement products in patients suffering with acute gastroenteritis. When combined with added fluids they can be used in patients with diarrhea without dehydration. This includes traveler's diarrhea and similar acute bacterial gut infections. In alternative embodiments, the compositions and purgatives of the invention are also gluten free and therefore acceptable to those with celiac disease.

In alternative embodiments, the contained xylose and/or other minimally degradable sugar(s) (being relatively inert as opposed to glucose) in compositions of the invention is particularly important in orthostatic lavage for colonoscopy as it will help to avoid fermentation and volatile explosive gas production (e.g. methane and hydrogen). The importance of this is that the potential of an explosion during diathermy polypectomy is reduced.

In alternative embodiments, an aim of the present invention is to replace lost sodium as well as water resulting from bowel preparation in intact epithelial cells devoid of toxin-induced block such as with cholera toxin Na—K ATPase pump. In alternative embodiments, the use of hypertonic solutions gives an opportunity to restore the osmotic equilibrium, which is altered by the induced water intoxication following replacement of fluid without electrolytes in patients undergoing some of the established bowel preparation protocols.

In alternative embodiments of methods for inducing purgation of the colon in a patient, a composition of the invention is provided in the form of a sachet which includes flavoring. The contents (typically weighing about 25 g) when mixed with water, preferably heated, in a quantity of 200-500 mls (1-10 ml/kg) will form a palatable soup, which may be cool or heated to form a hypertonic preparation with an osmolarity >350 mosm/l. In alternative embodiments, after consuming the above purgative dose, the patient will be instructed to ingest cool water at least 3 times the volume, or in an adult greater than 750-1000 mls of cool water.

In alternative embodiments, compositions of this invention are useful for colonoscopic lavage, as simple purgatives or in electrolyte replacement therapy, as preparations or an enhancement for barium enema, in X-ray computed tomography, computed tomography (CT scan) or computed axial tomography (CAT scan), for e.g., a “virtual colonoscopy” or other procedure, and also in the preparation and/or enhancement for other diagnostic, radiological or imaging applications, including CT scanning or equivalents, diagnostic sonography (ultrasonography), magnetic resonance imaging (MRI), nuclear magnetic resonance imaging (NMRI), or magnetic resonance tomography (MRT), and/or echocardiograms and the like.

### Bisoxatin

In alternative embodiments, the invention provides compositions comprising a bisoxatin (or 2,2-bis(4-hydroxyphenyl)-2H-benzo[b][1,4]oxazin-3(4H)-one), or bisoxatin acetate, or equivalent, including e.g., or a LAXONALIN™, a MARATAN™, a TALSIS™, or a TASIS™, or an equivalent.

In alternative embodiments, a formulation or composition of the invention comprises between about 10 mg to about 0.5, 1, 2, 2.5, 3, 3.5, 4, 4.5 or 5 or more grams of bisoxatin, or between about 0.5 and 5 grams (g) of bisoxatin, or between about 75, 80, 85, 90 or 100 mg to about 150 to 200 mg (e.g., for a normal patient) bisoxatin, or between about 100, 110, 120, 130, 140 or 150 mg to about 1, 2, 3, 4, 4.5 or 5 grams (g) or more bisoxatin (e.g., for a constipated patient).

### Contrast Media or Agents

In alternative embodiments, contrast media is added to a composition or formulation of the invention, or is used in conjunction with (e.g., simultaneously, before or after) administration of a composition or formulation of the invention. In alternative embodiments, contrast media or agent used to practice the invention include e.g., barium or iodine products, diatrizoate (e.g., HYPAQUE 50™), metrizoate (e.g. ISOPAQUE 370™), ioxalgate (e.g., HEXABRIX™), iopamidol (e.g., ISOVUE 370™), iohexol (e.g., OMNIPAQUE 350™), ioxilan (e.g., OXILAN 350™), iopramide (e.g., ULTRAVIST 370™), iodixanol (e.g., VISIPAQUE 320™) and/or a diatrizoic acid or its anionic form diatrizoate (also known as amidotrizoic acid, or 3,5-diacetamido-2,4,6-triiodobenzoic acid; e.g., HYPAQUE™, GASTROGRAFIN™, UROGRAFINT™).

In one embodiment, increasing the osmotic content of compositions or formulations of the invention, e.g., as capsules or tablets, will assist in their purgative effect. In one embodiment, diatrizoic acid or its anionic form diatrizoate or equivalents are used to increase the osmolarity of compositions or formulations of the invention (diatrizoic acid or its anionic form diatrizoate are high-osmolality contrast agents, having osmolality ranges from approximately 1500 mOsm/kg (50% solution) to over 2000 mOsm/kg (76% solution)). In one embodiment, nanoparticle agglomerates of diatrizoic acid (or its anionic form diatrizoate, or equivalents) are used in a composition or formulation of the invention, e.g., equivalent to the diatrizoic acid-containing nanoparticles formulated as inhalable microparticles, see e.g., El-Gendy, et al. (2010) Int. J. Pharm. 391(1-2): 305-312. In one embodiment, HYPAQUE™ sodium (diatrizoate sodium, USP) is used, e.g., as a sodium 3,5-diacetamido-2,4,6-triiodobenzoate having 59.87 percent iodine; it is available as a powder.

In alternative embodiments, a small content is placed into compositions or formulations of the invention, e.g., a tablet or capsule or equivalent; and in alternative embodiments, a sufficient amount of contrast medium e.g., diatrizoic acid or its anionic form diatrizoate, is added to increase the purgation effect and optionally also provide contrast to visualize the bowel, e.g., on an X-ray or a computed tomography (CT scan) or computed axial tomography (CAT scan) or equivalents; or compositions or formulations of the invention with contrast agents can be used with, to enhance or in preparation for a diagnostic, radiological or imaging application, including CT scanning or equivalents, diagnostic sonography (ultrasonography), magnetic resonance imaging (MRI), nuclear magnetic resonance imaging (NMRI), or magnetic resonance tomography (MRT), and/or echocardiograms and the like.

In alternative embodiments, compositions or formulations of the invention with contrast agents also are used as electrolyte replacement lavage solutions for acute gastrointestinal infections, for symptomatic improvement in those patients suffering from either acute or chronic constipation and related symptoms.

### Additional Optional Ingredients

Dyes, Vital Stains, Markers of Colonic or Rectal Mucosal Pathology

In alternative embodiments, dyes, vital stains or markers of mucosal pathology, e.g., a hexaminolevulinate, are added to a composition of the invention, or used to practice a method of the invention. In alternative embodiments, hexaminolevulinate, or CYSVIEW™, or hexaminolevulinate HCl, or equivalent, is added to a composition of the invention, e.g., a capsule or tablet, which can be ingested late in the preparation or dosage regimen. In alternative embodiments, compositions or formulations of the invention comprising a hexaminolevulinate or equivalent are used for fluorescence endoscopy for e.g., detection and treatment of polyps, premalignant and/or malignant lesions, including a hexaminolevulinate-based photodetection of rectal polyps, premalignant and/or malignant lesions, adenoma and cancers.

In alternative embodiments, the amount required can be between about 5 mg and 500 gm, or about 100 gm. Due to a large quantity of hexaminolevulinate passing in the colon, a larger volume can therefore be included to increase attachment to polyps. In some embodiments, only a small volume of hexaminolevulinate is required, and it will take up no greater volume than about 2 of the 900 mg capsules (e.g., 1.8 gm).

In alternative embodiments, in addition to or with hexaminolevulinate, or as an alternative to hexaminolevulinate, other markers of colonic or rectal mucosal pathology can be used. In alternative embodiments, compositions and formulations of the invention can comprise: delayed release methylene blue, including the MMX format of colonic-released methylene blue, which can stain the normal mucosa yet polyps do not stain and become more clearly visible. In alternative embodiments, any dye or vital stain or marker can be used in this preparation or with any composition and formulation of the invention, or to practice a method of the invention, including, e.g., one or more of the following: Curcumin (i) Riboflavin (ii) Riboflavin-5′-phosphate, Tartrazine, Quinoline Yellow, Sunset Yellow, FCF OrangE, Yellow S, Cochineal, Carminic acid, Carmines, Azorubine, Carmoisine, Ponceau 4R, Cochineal Red A, Allura Red AC, Patent Blu EV, Indigotine, Indigo carmine, Brilliant Blue FCF, Chlorophylls and chlorophyllins, Copper complexes of chlorophylls and chlorophyllins, Green S, Plain caramel, Brilliant Black BN, Black PN, Vegetable carbon, Brown HT, Carotenes, Lutein, Beetroot Red, betanin, Anthocyanins, Calcium carbonate, Titanium dioxide, Iron oxides and hydroxides, Amaranth, Brown FK, Erythrosine, Lithol Rubine BK and/or Red 2G or equivalents or any combination thereof.

In alternative embodiments, dyes or vital stain can be used with any composition and formulation of the invention, or to practice a method of the invention, include, e.g., acid fuchsine, Alba red, Alizarin cyanine green F, Alizurol purple S5, Allura Red AC, Alphazurine FGBrilliant lake red R, Dibromofluorescein, Diiodofluorescein, Eosine, Erythrosine yellowish Na, Fast green FCF, Flaming red, Fluorescein, Helindone pink CN, Indanthrene blue, Lake bordeaux B, Lithol rubin B Ca, Naphthol yellow 5, Orange II, Phloxine B, Ponceau 5X, Pyranine concentrated, Quinizarinegreen 5S, Tetrabromo-fluorescein, Tetrachlorotetrabromo fluorescein, Toney red, Uranine, Alcian Blue, Anazolene Sodium, Brilliant Green, Cantaxanthin, Carthamin, Citrus Red 2, Evan's Blue, Fast Green FCF, Indocyanine Green, Methyl Blue, Methylene Blue, N-(p-Methoxyphenyl)-p-phenylenediamine, Ponceau 3R, Ponceau SX, Pyranine, Rhodamine B, Saunders Red, Sudan Black B, Sulphan Blue, Tolonium Chloride, and/or Vital Red or equivalents or any combination thereof.

Surfactants

In alternative embodiments, a surfactant is added into a composition or formulation of the invention, or used to practice a method of the invention. In alternative embodiments, simethicone (or any mixture of polydimethylsiloxane and silica gel), dimethicone or similar or equivalent surfactant is added into a composition or formulation of the invention; optionally between about 5 mg and 450 mg can be added.

Lubricants

In alternative embodiments, a lubricant is added into a composition or formulation of the invention, or used to practice a method of the invention. The addition of lubricants such as glycerol or silicone to the formulation can help with a colonoscope insertion and facilitation within the performance of the colonoscopy.

Biofilm Disrupting Compounds

In alternative embodiments, biofilm disrupting compounds added into a composition or formulation of the invention, or used to practice a method of the invention. In alternative embodiments, disrupting biofilms are used to separate from the colonic mucosa an adherent polysaccharide/DNA—containing layer, the so-called “biofilm”, to achieve a cleaner and/or more easily visualized or stained mucosa. In alternative embodiments, bisoxatin itself is used, it has such an action in-part, achieving a cleaner caecum.

In alternative embodiments, other biofilm disrupting components or agents also can be used, e.g., enzymes such as deoxyribonuclease (DNase), N-acetylcysteine, alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7 (a small lytic peptide, see e.g., Kharidia (2011) J. Microbiol. 49(4):663-8, Epub 2011 Sep 2), Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones and/or macrolide antibiotics or any combination thereof.

In alternative embodiments, biofilm disrupting components or agents are administered with a formulation or composition of the invention, e.g., are administered throughout or concentrated at the end of a capsule bowel prep ingestion so as to disrupt the biofilm most just before the colonoscopy.

Bisacodyl

In alternative embodiments, compositions and formulations of the invention can further comprise a bisacodyl, or pyridin-2-ylmethanediyl)dibenzene-4,1-diyldiacetate, or 4,4′-(pyridin-2-ylmethylene) bis(4,1-phenylene) diacetate, or a bioequivalent diphenylmethane. In alternative embodiments, the bisacodyl or bioequivalent diphenylmethane is formulated at or less than about 25 mg, 24 mg, 23 mg, 22 mg, 21 mg, 20 mg, 19 mg, 18 mg, 17 mg, 16 mg, 15 mg, 14 mg, 13 mg, 12 mg, 11 mg, 10 mg, 9 mg, 8 mg, 7 mg, 6 mg, 5 mg, 4 mg, 3 mg, 2 mg or 1 mg or less, or are between about 1 and 25 mg per dosage (per unit dosage). In alternative embodiments, the bisacodyl or bioequivalent diphenylmethane is formulated at between about 1, 5, 10, 15, 20 or 25 mgm to about 100, 150, 200, 225 or 250 or more mgm per unit dosage.

In alternative embodiments the bisacodyl, or equivalent is administered at a dosage of between about 1 to 360 mgm a day, or is administered at a dosage of 1.0, 2, 3, 4, 5, 6, 7, 8, 9, 10, 15, 20, 21, 22, 23, 24, 25, 30, 31, 32, 33, 34, 35, 36, 37, 38, 39, 40, 40, 45, 50, 55, 60, 70, 80, 90, 100, 125, 150, 175, 200, 225, 250, 275, 300, 325, 350 or 360 milligram (mg) a day. In alternative embodiments the unit dosage of the bisacodyl, or equivalent is between about 20 to 120 mgm per unit dosage, or the unit dosage is about 20, 21, 22, 23, 24, 25, 30, 31, 32, 33, 34, 35, 36, 37, 38, 39, 40, 40, 45, 50, 55, 60, 70, 75, 80, 90, 100, 110, 115, 120 or 125 mgm per unit dosage.

In alternative embodiments, the bisacodyl is DULCOLAX™, DUROLAX™, FLEET™, ALOPHEN™, CORRECTOL™, and/or the bisoxatin is LAXONALIN™, MARATAN™, TALSIS™, TASIS™.

### Unit Dosage Forms and Formulations and Delivery Vehicles

In alternative embodiments, a composition is manufactured, labeled or formulated as a liquid, a suspension, a spray, a gel, a geltab, a semisolid, a tablet, or sachet, a capsule, a lozenge, a chewable or suckable unit dosage form, or any pharmaceutically acceptable formulation or preparation. In alternative embodiments, a composition of the invention is incorporated into a food, a feed, a drink, a nutritional or a food or feed supplement (e.g., liquid, semisolid or solid), and the like.

For example, a composition of the invention can be manufactured, labeled or formulated as an orally disintegrating tablet as described e.g., in U.S. Pat. App. Publication No. 20100297031. A composition of the invention can be a polyol/thickened oil suspension as described in U.S. Pat. Nos. 6,979,674; 6,245,740. A composition of the invention can be encapsulated, e.g., encapsulated in a glassy matrix as described e.g., in U.S. Pat. App. Publication No. 20100289164; and U.S. Pat. No. 7,799,341. A composition of the invention can be manufactured, labeled or formulated as an excipient particle, e.g., comprising a cellulosic material such as microcrystalline cellulose in intimate association with silicon dioxide, a disintegrant and a polyol, sugar or a polyol/sugar blend as described e.g., in U.S. Pat. App. Publication No. 20100285164. A composition of the invention can be manufactured, labeled or formulated as an orally disintegrating tablet as described e.g., in U.S. Pat. App. Publication No. 20100278930. A composition of the invention can be manufactured, labeled or formulated as a spherical particle, as described e.g., in U.S. Pat. App. Publication No. 20100247665, e.g., comprising a crystalline cellulose and/or powdered cellulose. A composition of the invention can be manufactured, labeled or formulated as a rapidly disintegrating solid preparation useful e.g. as an orally-disintegrating solid preparation, as described e.g., in U.S. Pat. App. Publication No. 20100233278. A composition of the invention can be manufactured, labeled or formulated as a solid preparation for oral application comprising a gum tragacanth and a polyphosphoric acid or salt thereof, as described e.g., in U.S. Pat. App. Publication No. 20100226866. A composition of the invention can be manufactured, labeled or formulated using a water soluble polyhydroxy compound, hydroxy carboxylic acid and/or polyhydroxy carboxylic acid, as described e.g., in U.S. Pat. App. Publication No. 20100222311. A composition of the invention can be manufactured, labeled or formulated as a lozenge, or a chewable and suckable tablet or other unit dosage form, as described e.g., in U.S. Pat. App. Publication No. 20100184785. A composition of the invention can be manufactured, labeled or formulated in the form of an agglomerate, as described e.g., in U.S. Pat. App. Publication No. 20100178349. A composition of the invention can be manufactured, labeled or formulated in the form of a gel or paste, as described e.g., in U.S. Pat. App. Publication No. 20060275223. A composition of the invention can be manufactured, labeled or formulated in the form of a soft capsule, as described e.g., in U.S. Pat. No. 7,846,475, or U.S. Pat. No. 7,763,276.

In one embodiment, a composition of the invention is incorporated into a food, a feed, a drink, a nutritional or a food or feed supplement (e.g., liquid, semisolid or solid), and the like, as described e.g., in U.S. Pat. App. Publication No. 20100178413. In one embodiment, a composition of the invention is incorporated into (manufactured as) a beverage as described e.g., in U.S. Pat. No. 7,815,956. For example, a composition of the invention is incorporated into a yogurt, an ice cream, a milk or milkshake, a “frosty”, “snow-cone”, or other ice-based mix, and the like.

The polyols used in compositions of the invention can be micronized polyols, e.g., micronized polyols, e.g., as described e.g., in U.S. Pat. App. Publication No. 20100255307, e.g., having a particle size distribution (d₅₀) of from 20 to 60 μm, and a flowability below or equal to 5 s/100 g, or below 5 s/100 g.

The invention will now be described with reference to the following examples which should not be construed as limiting on the present invention.

### Example 1

A 57 year old female was undergoing preparation for surveillance colonoscopy due to positive family history for cancer. She was offered a bowel preparation of the invention containing bisoxatin, sodium, potassium and magnesium electrolytes, as well as erythritol in encapsulated format as described above. The last 10 capsules contained methylene blue in enteric-coated capsules. The patient achieved excellent purgation. The entire colonic mucosa at colonoscopy was essentially free of any attached stool matter. The mucosa was quite blue in colour and created a ‘dark tunnel’ appearance akin to pseudomelanosis coli. However, two elevated areas resembling polyps found between haustrations in the ascending colon failed to stain to the same extent and stood out from the deeper blue mucosal staining, and upon removal with cold biopsy forceps were documented to be adenomatous polyps.

### Example 2

Five patients undergoing colonoscopy (two constipated) were given the exemplary “bisoxatin capsule prep” of the invention containing electrolytes, erythritol and biofilm-disrupting N-acetyl cystine [NAC] 300 mg per capsule in the last 4 capsules to be ingested. At colonoscopy, the usually generally clean colonic mucosa appeared shiny and more free of even specks of faeces especially in the caecum and ascending colon where constipated patients often show evidence of some stool attachment. In addition, the remaining liquefied fluid had no particulate matter, was low in volume and was easy to aspirate through the colonoscope channel. It was the impression of the colonoscopists that the mucosa achieved a higher level of cleansing due to the NAC.

### Example 3

In seven patients powdered dimethicone in a total mass of 5 mg was evenly added across the 33 capsules of the exemplary bisoxatin/electrolyte-containing capsules of the invention. Although in the standard bisoxatin-containing bowel preparation the mucosa is generally cleansed quite well, the remaining fluid may contain bile salts which may interfere with visibility by forming “foam” and visible bubbling which needs to be washed away. This can be quite a frequent phenomenon. The repeated washing and aspiration slows down progress of colonoscopy and reduces visibility. In the patients described in this example there was total abolition of formation of bile salt “foaming”. The minimum amount of dimethicone required to achieve this may well be smaller than 5 mg. The use of simethicone in other patients achieved a similar result but required a liquid format of simethicone added to the ingested fluid during bowel preparation since no powder simethicone was available at this stage.

### Example 4

Two patients were known to suffer from mild constipation and frequent cramping during previous use of liquid pre-colonoscopy bowel preps [Glycoprep and Picoprep]. The new, exemplary encapsulated bowel prep of the invention comprising the above-described electrolytes bisoxatin and erythritol had added Gastrografin [500 mg over the last 10 capsules]. The patients appeared to have liquid diarrhoea of greater volume and frequency, exceeding 15 liquid stools prior to colonoscopy. Nonetheless, neither patient experienced cramping and at colonoscopy visualisation appeared excellent. The patients both were convinced this new purgative was responsible for preventing prep-associated cramping.

A number of embodiments of the invention have been described. Nevertheless, it will be understood that various modifications may be made without departing from the spirit and scope of the invention. Accordingly, other embodiments are within the scope of the following claims.

1. A composition comprising:
between about 2 to about 37 grams of sodium sulfate,
at least one water-soluble potassium salt;
erythritol;
and a bisoxatin (or 2,2-bis(4-hydroxyphenyl)-2H-benzo[b][1,4]oxazin-3(4H)-one), or bisoxatin acetate, or equivalent.

2. (canceled)

3. The composition of claim 1, wherein the weight of the water-soluble potassium salt in the composition is from about 0.05 to about 1 times the weight of the sodium sulfate in the composition.

4. The composition, pharmaceutical composition or formulation of claim 1, wherein the water-soluble potassium salt is selected from the group consisting of a potassium sulfate, a potassium chloride and a potassium tartrate.

5. The composition of claim 1, further comprising a sodium picosulfate, a sodium sulphate, a bisacodyl or a combination thereof.

6. The composition of claim 1, further comprising at least one composition or additive selected from the group consisting of a flavoring ingredient, citrate, lactate, acetate, a trace element and a nutritional element.

7. The composition of claim 1, wherein the composition is formulated as a liquid, a fluid, or a soup or soup-like composition.

8. (canceled)

9. The composition of claim 1, further comprising at least one water-soluble magnesium salt.

10. The composition of claim 1, further comprising one or more compositions or additives selected from the group consisting of a citrate, a lactate, an acetate, a calcium, a zinc, a Vitamin B complex, a thiamine, a Vitamin A, a Vitamin C, a Vitamin E, a folic acid and a biotin.

11. The composition of claim 1, in the form of a plurality of tablets, gel caps or capsules.

12. The composition of claim 9, wherein
the at least one water-soluble magnesium salt comprises a magnesium sulfate, a magnesium citrate or a magnesium phosphate.

13. A method of inducing a pre-surgical lavage of the colon of a patient in need thereof, comprising administering to the patient the composition of claim 1.

14. A method of inducing purgation of the colon of a patient in need thereof, comprising administering to the patient the composition of claim 1.

15-16. (canceled)

17. The composition of claim 1, further comprising one or more of a contrast media, a barium or an iodine comprising composition or product, a diatrizoate, a metrizoate, an ioxalgate, an iopamidol, an iohexol, an ioxilan, a iopramide, an iodixanol, and/or a diatrizoic acid or its anionic form diatrizoate.

18. The composition of claim 1, further comprising a dye or vital stain or marker.

19. The composition of claim 1, further comprising a surfactant.

20. The composition of claim 1, further comprising a lubricant.

21-23. (canceled)

24. The composition of claim 1, formulated as a sachet or a plurality of sachets.

25. The composition of claim 1, wherein the ratio of the potassium salt to sodium salt in the composition is from about 1:1 to about 1:8.

26. The composition of claim 1, wherein the at least one potassium salt is present in an amount of from about 0.5 gram to 5 grams per unit dose.

27. The composition of claim 9, wherein the weight of magnesium ions to the weight of sodium ions in the composition is from about 1:5 to about 5:1.

28. The composition of claim 9, wherein the at least one magnesium salt is present in an amount of from about 1 gram to about 10 grams per unit dose.

29. A pharmaceutical composition, comprising:
between about 2 to about 37 grams of sodium sulfate,
at least one water-soluble potassium salt;
at least one water-soluble sugar, a water-soluble degradable sugar, or a minimally degradable sugar; and
bisoxatin (or 2,2-bis(4-hydroxypheny)-2H-benzo(b)[1,4]oxazin-3(4H)-one), or bisoxatin acetate.
